(19) United States
(12) Patent Application Publication
(10) Pub. No.: US 2015/0272911 A1
(43) Pub. Date: Oct. 1, 2015

(54) Use of AMPK Inducers and Metformin for the Treatment of Fibromyalgia

(71) Applicant: Universidad de Sevilla, Sevilla (ES)

(72) Inventors: Mario David Cordero Morales, Sevilla (ES); Pedro Bullon Fernandez, Sevilla (ES); Elisabet Alcocer Gomez, Sevilla (ES)

(73) Assignee: Universidad de Sevilla, Sevilla (ES)

(21) Appl. No.: 14/264,354

(22) Filed: Apr. 29, 2014

(30) Foreign Application Priority Data
Mar. 27, 2014 (ES) P201400296

(51) Int. Cl. A61K 31/155

(52) U.S. Cl. CPC A61K 31/155

(57) ABSTRACT

The present invention relates to the use of metformin, metformin derivatives, compounds containing metformin and compounds that induce the activation of AMPK phosphorylation for the treatment of fibromyalgia, reducing pain and depression, as well as dysfunctional biological parameters.

The primary sectors of application are the pharmaceutical and medical sectors, through the development of tablets with lower doses than those currently used for these patients.

### OBJECT OF THE INVENTION

The present invention relates to the use of metformin, metformin derivatives, compounds containing metformin and compounds that induce the activation of AMPK phosphorylation for the treatment of fibromyalgia, reducing pain and depression, as well as dysfunctional biological parameters.

The primary sectors of application are the pharmaceutical and medical sectors, through the development of tablets with lower doses than those currently used for these patients.

### STATE OF THE ART

Fibromyalgia is a chronic generalised pain syndrome accompanied by other symptoms such as depression, anxiety, fatigue or sleep alterations. Its diagnosis is made on the basis of the classification criteria established by the American College of Rheumatology (ACR) (Wolfe et al., 1990), and it does not show biochemical alterations in any routine diagnostic test (Yunus et al., 1981), for which reason there is a need to find new diagnostic markers for the disease. The prevalence of fibromyalgia in industrialised countries ranges between 2% and 4% of the population, and is 11 times more frequent in women than in men (Lawrence et al., 2008); however, the etiopathogenic mechanisms of the disease are still unknown.

In recent years, new research has provided important data to understand the physiopathological mechanisms of fibromyalgia. It has been determined that various alterations in the metabolism, transport and reuptake of serotonin (Cordero et al., 2011), as well as in the inflammatory cytokines, may play a role in the pathogenesis of fibromyalgia (Cordero et al., 2013). There is no medicament that has shown high efficacy in controlling all the symptoms of fibromyalgia, and the most adequate treatment for this disease is a rational, individualised combination of drugs. At present, the most widely used medications to relieve the symptoms of fibromyalgia include analgesics, anxiolytic agents, tricyclic and other types of antidepressants, muscle relaxants, dual serotonin/noradrenaline reuptake inhibitors, non-benzodiazepine sleep inducers, modifiers of dopamine metabolism, etc. However, although there is a large number of prescription drugs aimed at relieving the symptoms of fibromyalgia, these are only highly effective in a low percentage of patients (about 20% of them improve considerably), but are accompanied by numerous secondary effects.

Developing a new drug may take between 10 and 17 years, with a cost of over Euro 800 million. A quicker route to find new treatments against a disease involves discovering potential new functions in old drugs already authorised for other diseases. This is the case of metformin, one of the most popular antidiabetic agents. The present invention is based on a new indication for this old drug. Fibromyalgia presents a number of biological characteristics, such as oxidative stress, low levels of ATP, mitochondrial dysfunction, inflammation, low mitochondrial biogenesis, and low expression of AMPK, associated with clinical symptoms like pain, fatigue and depression (Cordero et al., 2013a, 2013b). All the alterations described in fibromyalgia have been restored in the mononuclear cells and fibroblasts of patients with fibromyalgia by means of treatment with metformin. Moreover, metformin has proven to have a strong analgesic effect in other chronic pain pathologies, such as neuropathic pain (Russe et al., 2013) or adiposis dolorosa (Labuzek et al., 2013). In this regard, we have been able to verify that, following the inhibition of AMPK by compound c (a specific inhibitor of AMPK activation) in mice, these presented symptoms typical of fibromyalgia, such as pain or depression. Following the treatment with metformin, these animals showed a recovery in the symptoms (See figures).

On the other hand, another parameter found in patients with fibromyalgia is activation of the inflammasome complex (Cordero et al., 2013c), which is related to pain in mice following the inhibition of AMPK (See figures). The inflammasome is a group of proteins that participate in the intracellular detection and eradication system, and is an essential aspect of the innate immune system. The animal models related to molecular alterations of the inflammasome complex are genetic models, and there are no references about their relation to generalised pain. Metformin has demonstrated inhibition of the inflammasome in the cells of patients with fibromyalgia, as well as in mice with inhibited AMPK.

Therefore, metformin acts by activating AMPK, with the consequent reductions in oxidative stress, mitochondrial dysfunction and inflammation, as well as activation of ATP synthesis and mitochondrial biogenesis in fibromyalgia, and in controlling the symptoms associated with fibromyalgia. Currently, new functions of metformin have been described thanks to these capacities, which have been useful in the treatment of other pathologies different from type II Diabetes, such as the metabolic syndrome (WO/2013/159190), type I Myotonic Dystrophy (20130085169), autoimmune diseases (1020130031229), Cancer (20120220664).

The fact that metformin is an already-commercialised drug is highly significant, since it has satisfactorily passed phase I clinical trials and has been approved for use in humans due to its safety. Therefore, if it were to be used for another therapeutic indication, the clinical development phase would be achieved in a much shorter period of time than for traditional models based on new chemical entities with a long pre-clinical development. We propose to use metformin as a drug for the treatment of fibromyalgia, which, according to our data, makes it possible to use it at reduced doses as compared to its use in type II Diabetes.

Metformin has a broad variety of forms for sale which have proven to be safe for patients:

COMPETACT Film-coated tabl. 15 mg/850 mg
DIANBEN Film-coated tabl. 850 mg
DIANBEN Powder for sol. in sachets 850 mg
DIANBEN Powder for oral sol. in sachets 1000 mg
EFFICIB Coated tabl. 50 mg/1000 mg
EUCREAS Film-coated tabl. 50 mg/1000 mg
EUCREAS Film-coated tabl. 50 mg/850 mg
GLUBRAVA Film-coated tabl. 15 mg/850 mg
ICANDRA Film-coated tabl. 50/1000 mg
ICANDRA Film-coated tabl. 50/850 mg
JANUMET Film-coated tabl. 50 mg/1000 mg
JENTADUETO Film-coated tabl. 2.5/850 mg
KOMBOGLYZE Film-coated tabl. 2.5/1000 mg
KOMBOGLYZE Film-coated tabl. 2.5/850 mg
METFORMIN ACTAVIS Film-coated tabl. 850 mg
METFORMIN ADLER-APOTHEKE Film-coated tabl. 1000 mg
METFORMIN ADLER-APOTHEKE Film-coated tabl. 850 mg
METFORMIN ALDO-UNION Film-coated tabl. 850 mg
METFORMIN ALMUS EFG Film-coated tabl. 850 mg
METFORMIN APOTEX Film-coated tabl. 850 mg
METFORMIN AUROBINDO Film-coated tabl. 850 mg
METFORMIN BLUEFISH Film-coated tabl. 850 mg
METFORMIN CINFA EFG Film-coated tabl. 850 mg
METFORMIN COMBIX Film-coated tabl. 850 mg
METFORMIN DAVUR Film-coated tabl. 850 mg
METFORMIN DRAGENOPHARM Film-coated tabl. 1000 mg
METFORMIN DRAGENOPHARM Film-coated tabl. 850 mg
METFORMIN EDIGEN Film-coated tabl. 850 mg
METFORMIN KERN PHARMA Film-coated tabl. 1000 mg
METFORMIN KERN PHARMA EFG Film-coated tabl. 850 mg
METFORMIN MYLAN Film-coated tabl. 1000 mg
METFORMIN MYLAN Film-coated tabl. 850 mg

### BIBLIOGRAPHY

Cordero M D, de Miguel M, Moreno-Fernández A M. Mitochondrial dysfunction in fibromyalgia and its implication in the pathogenesis of disease. Med Clin (Barc). 2011 Mar. 12; 136(6): 252-6.
Cordero M D, Díaz-Parrado E, Carrión A M, Alfonsi S, Sánchez-Alcazar J A, Bullón P, Battino M, de Miguel M. Is inflammation a mitochondrial dysfunction-dependent event in fibromyalgia? Antioxid Redox Signal. 2013a Mar. 1; 18(7): 800-7.
Cordero M D, Alcocer-Gómez E, de Miguel M, Culic O, Carrión A M, Alvarez-Suarez J M, Bullón P, Battino M, Fernández-Rodríguez A, Sánchez-Alcazar J A. Can Coenzyme Q10 Improve Clinical and Molecular Parameters in Fibromyalgia? Antioxid. Redox. Signal. 2013b. 19: 1356-61.
Cordero M D, Alcocer-Gómez E, Culic O, Carrión A M, de Miguel M, Díaz-Parrado E, Perez-Villegas E M, Bullón P, Battino M, Sánchez-Alcázar J A. NLRP3 Inflammasome is activated in Fibromyalgia: the effect of Coenzyme Q10. Antioxid Redox Signal. 2012c. doi:10.1089/ars.2013.5198.
Labuzek K, Liber S, Suchy D, Okopieå B A. A successful case of pain management using metformin in a patient with adiposis dolorosa. Int J Clin Pharmacol Ther. 2013 June; 51(6): 517-24.
Lawrence R C, Felson D T, Helmick C G, Arnold L M, Choi H, Deyo R A, et al. Estimates of the prevalence of arthritis and other rheumatic conditions in the United States. Part II. Arthritis Rheum 2008; 58: 26-35.
Russe O Q, Möser C V, Kynast K L, King T S, Stephan H, Geisslinger G, Niederberger E. Activation of the AMP-Activated Protein Kinase Reduces Inflammatory Nociception. J Pain. 2013 Nov. 14(11): 1330-40.
Wolfe F et al. The American College of Rheumatology 1990 Criteria for the Classification of Fibromyalgia. Report of the Multicenter Criteria Committee. Arthritis Rheum 1990; 33: 160-172.
Yunus M, Masi A T, Calabro J J, et al. Primary fibromyalgia (fibrositis): clinical study of 50 patients with matched normal controls. Semin Arthritis Rheum 1981; 11: 151-71.

### DESCRIPTION OF THE FIGURES

FIG. 1 shows the levels of intracellular ATP synthesis in the blood mononuclear cells (BMCs) of patients with fibromyalgia following the treatment with metformin.

FIG. 2 shows the levels of production of oxygen free radicals (ROS) in the BMCs of patients with fibromyalgia following the treatment with metformin.

FIG. 3 shows the IL-1β (a) and IL-18 (b) levels in serum in supernatants of the cells of patients treated with metformin (pg/ml).

FIG. 4 shows the levels of protection of metformin following exposure of the fibroblasts of patients with fibromyalgia to hydrogen peroxide. % cell death.

FIG. 5 shows the levels of intracellular ATP synthesis in the blood mononuclear cells (BMCs) of the experimental model by the inhibition of AMPK phosphorylation following the treatment with metformin.

FIG. 6 shows the levels of citrate synthase (mitochondrial mass) in the experimental model by inhibition of AMPK phosphorylation (Enzyme activity) and following the treatment with metformin. FIG. 7 shows the levels of serum lipid peroxidation as markers of oxidative stress in the experimental model by AMPK inhibition and treatment with metformin (nmol/ml).

FIG. 8 shows the IL-1β (a) and IL-18 (b) serum levels in the sera of mice which had AMPK phosphorylation inhibited and following the treatment with methofibromyalgiairna (pg/ml).

FIG. 9 shows the nociceptive evaluation of the model (latency in seconds). Percentage of animals with pain.

FIG. 10 shows the evaluation of depression (immobilisation time, seconds). Percentage of animals with the symptom.

### DESCRIPTION OF THE INVENTION

Fibromyalgia is a disease characterised by chronic pain which is highly prevalent, affecting almost 5% of the world population (Lawrence et al., 2008). The study of the molecular mechanisms of pain and, more specifically, of fibromyalgia, is one of the challenges for science and, consequently, for the pharmaceutical industry, given its mission to develop increasingly more effective drugs to approach it. Studies of biological samples from patients with fibromyalgia have shown numerous alterations associated with the physiopathology of fibromyalgia, as described above. Metformin has demonstrated a significant potential for the restoration of these alterations; for this reason, it has been the object of new functions in other pathologies, for which it has generated patents, listed in the preceding section.

Our invention offers a new use for an already-known drug, which has passed the clinical phases that demonstrate its safety, and which, at reduced doses as compared to those used in type II Diabetes, offers a new, effective treatment for fibromyalgia. Its placing in the market would take a short period of time, since it would only be necessary to prepare lower-dose tablets, and its effectiveness in relieving pain in fibromyalgia is accompanied by an improvement in other symptoms, eliminating the secondary effects attributed to the commonly used treatments for fibromyalgia and with low effectiveness, such as oxidative stress or mitochondrial dysfunction. Moreover, metformin and compounds that induce AMPK are small molecules; therefore, they have ideal characteristics for conjugation in vehicles such as nanoparticles or liposomes, in order to target different tissues. On the other hand, they may be applied by means of injectables to patients with digestive problems.

Metformin has demonstrated its safety in patients with type II diabetes mellitus, as well as new functions that are currently being developed, due to its effect on the activation of metabolism, via AMPK, which induces weight loss in patients. The minimum usage dose is one tablet a day of any of its commercial forms, which are 850 mg or 1000 mg. In our studies, we have verified that, at levels below the normal usage dose, the effectiveness is significant. This effect may be extrapolated to any composition containing metformin, as well as inducers of AMPK phosphorylation, the latter being the main focus of the therapeutic mechanism of metformin and compounds that induce AMPK in fibromyalgia.

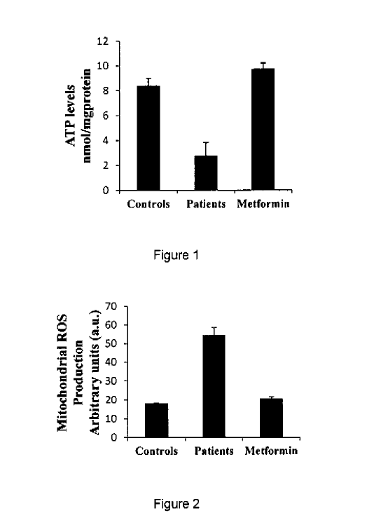

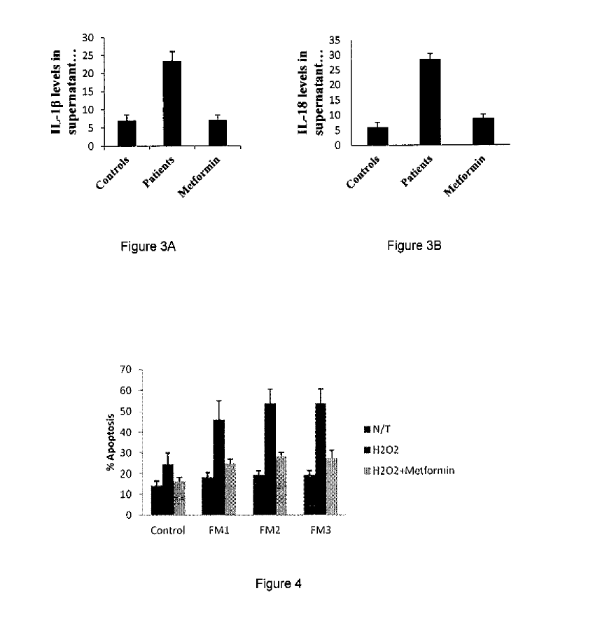

In order to study the role of AMPK phosphorylation in patients with fibromyalgia, AMPK activation was induced with metformin, a potent AMPK stimulator, in the BMCs of 20 patients. Following this treatment, the BMCs of the patients showed a significant recovery of ATP levels (FIG. 1) and a decrease in mitochondrial ROS (FIG. 2) and IL-1β and IL-18 interleukins of the inflammasome (FIG. 3), both of which are common alterations normally found in the BMCs of patients with fibromyalgia (Cordero et al., 2013b). On the other hand, metformin provides a protective effect to the cells of patients with fibromyalgia subjected to stressful factors, such as oxidative stress. The fibroblasts of patients with fibromyalgia show an increase in cell death when they are subjected to low doses of hydrogen peroxide, whereas, when they are subjected thereto and metformin is added, cell death was significantly reduced (FIG. 4).

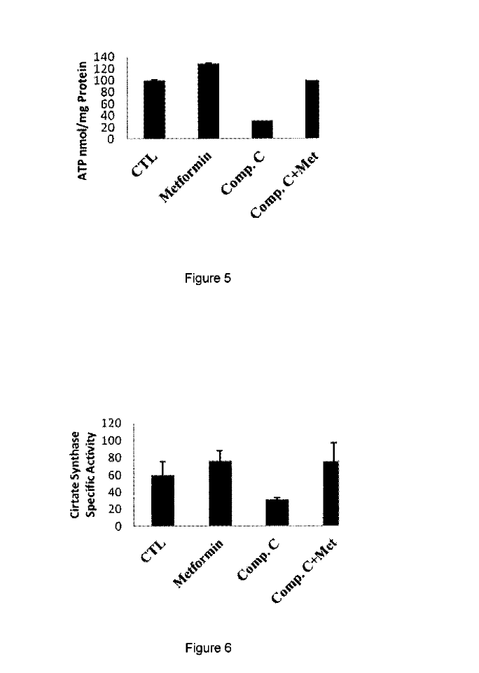

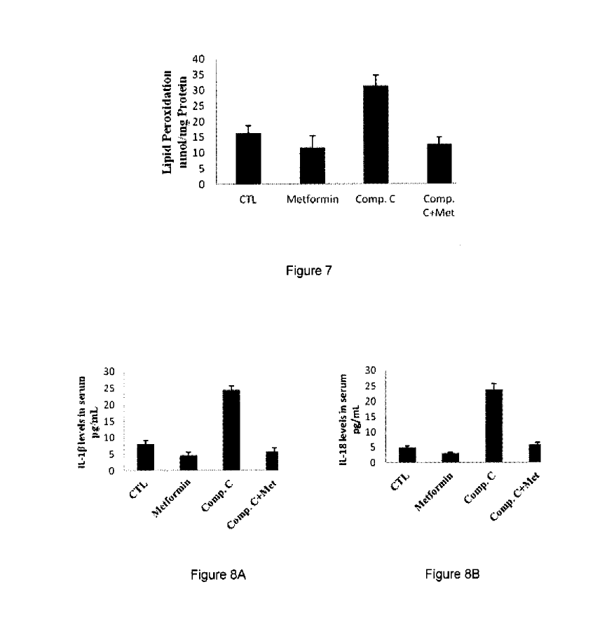

Metformin has also been proven to have an effect on the symptoms induced by a deficiency of AMPK activation. To this end, a model was developed in non-inbred mice of the Swiss strain, 2-3 months of age, by the induction of a deficiency of AMPK activation through the administration of compound C—(6-[4-(2-Piperidin-1-yl-ethoxy)-phenyl)]-3-pyridin-4-yl-pyrazolo [1,5-a]pyrimidine, a potent inhibitor of AMPK phosphorylation. The mice were injected by intraperitoneal route once a day, the final daily dose being 20 mg/kg/day. Another group of mice received the treatment with Compound C in addition to Metformin. The treatment is performed until the day the animals are sacrificed. The inhibition of AMPK induced alterations typical of fibromyalgia in the mice, which were restored by metformin, such as deficit of ATP (FIG. 5) and citrate synthase (mitochondrial mass) (FIG. 6), lipid peroxidation (FIG. 7) and activation of the IL-1β and IL-18 interleukins of the inflammasome (FIG. 8).

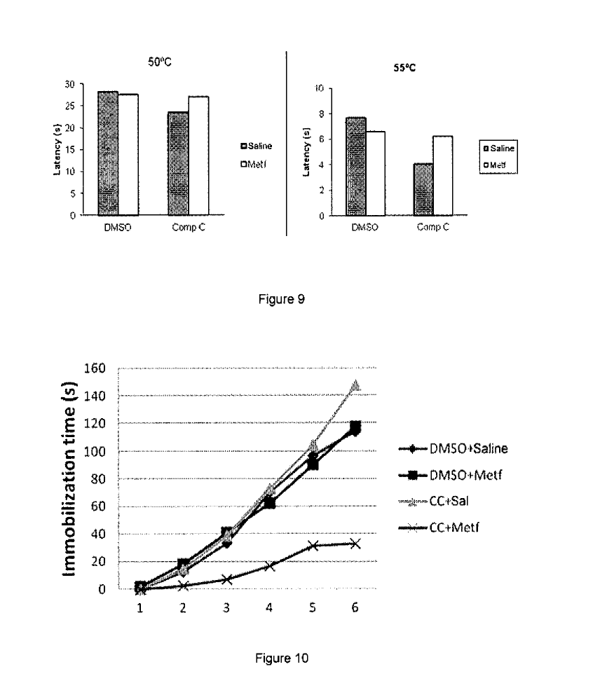

Following the first five days of administration of compound c, behavioural determinations were made in order to evaluate the symptoms. The hot-plate test shows a decrease in the response latency to the thermal stimulus at both non-painful temperatures (Temp.<50° C.) and painful temperatures (Temp.>50° C.), which reflects allodynia and hyperalgesia, respectively, an increase in pain in the mice treated with compound c (FIG. 9). Another symptom associated with pain in fibromyalgia is depression. In addition to showing generalised pain, when evaluated, the mice treated with compound c presented depression; using the tail suspension test, they showed greater paralysis throughout the test, a feature that is considered to reflect depression (FIG. 10).

### EMBODIMENT OF THE INVENTION

In order to evaluate the effect of metformin in in vitro models of fibromyalgia, we will use mononuclear cells isolated from patients with fibromyalgia and kept under culture conditions, with RPMI medium with 10% serum and 5% CO₂. Parallel to this, skin fibroblasts isolated from patients with fibromyalgia kept under culture with DMEM medium with 20% serum and 5% CO₂ will be used. These cultures will enable us to evaluate the toxicity of different doses of metformin and compounds that induce AMPK phosphorylation, through the quantification of apotosis and cell growth, by counting the nuclei of live cells. Once the ideal working dose has been found, below 5 mM, we will be able to quantify the protection against stressful stimuli through the effect of hydrogen peroxide, a very potent free radical. Using flow cytometry, we will evaluate the production of ROS before and after the treatment below 5 mM. Other parameters to be quantified at these doses will be ATP, by means of luminometry, citrate synthase as a marker of mitochondrial mass, by means of enzyme kinetics spectrophotometry, and inflammatory interleukins by means of ELISA.

In order to monitor the effect of metformin in an in vivo model of AMPK deficit, we will use mice that will be administered compound c, a specific inhibitor of AMPK phosphorylation, in time, and their behaviour will be characterised one week before starting the administrations of compound c and 7 days after the first administration. This experimental design will provide us with a view as to how metformin inhibits the effects induced by the deficit of AMPK and its influence on the behaviour of the mice, monitoring, with particular interest, those tests that show an alteration of nociception (hot plate, visceral pain [J Neurochem (2007) 103: 2629-39] and muscle pain [J Neurosci (2007) 27: 10000-6]). Prior to beginning the administration of compound c and metformin, and following the pain behaviour tests, other behavioural tests will be conducted which show other symptoms presented by patients with fibromyalgia:

Depression test: Tail suspension, forced swimming.

After finishing the treatment with compound c and metformin, the mice will be sacrificed and the following will be obtained:

Blood: The blood will be the starting-point to obtain serum or isolate leukocytes. The following will be measured in the serum: glucose, triglycerides, cholesterol, lactate, proteins, redox potential, protein oxidation, etc. The following will be measured in the leukocytes: ATP, inflammatory interleukins, oxidative stress, mitochondrial mass.

Striated skeletal muscle: The following will be measured in the muscle necropsies: ATP, inflammatory interleukins, mitochondrial activities, cytochrome c oxidase activity, and morphology and number of mitochondria.

In patients, it may be used to evaluate its effects on fibromyalgia using doses below those used for diabetes. Evaluation of the patients before and after the treatment with metformin between 200 and 400 mg a day will be performed by means of the fibromyalgia impact test, visual pain scales, Beck's depression test, etc.

1. A method of treatment of the fibromyalgia and the symptoms thereof which comprises administering to a patient an effective amount of inducers of AMPK phosphorylation, its derivatives or compositions comprising said inducers by means of tablets containing them at reduced doses, within a range of 200 to 400 mg a day.

2. The method according to claim 1, wherein the inducer of AMPK phosphorylation is metformin, its derivatives or compositions comprising metformin.

3. A method of treatment of the fibromyalgia and the symptoms thereof which comprises administering an effective amount of an effective amount of inducers of AMPK phosphorylation its derivatives or compositions comprising said inducers, by means of injectables, intravenous or intramuscular incorporated into a vehicle, such as nanoparticles or liposomes, for passive or active targeted therapy, the final daily dose ranging between 200 and 400 mg.

4. The method according to claim 3 wherein the inducer of AMPK phosphorylation is metformin, its derivatives or compositions comprising metformin.
